(19) United States
(12) Patent Application Publication
(10) Pub. No.: US 2013/0225929 A1
(43) Pub. Date: Aug. 29, 2013

(54) QUICK ATTACH LARYNGOSCOPE POWER GENERATOR DEVICE

(71) Applicant: Murray Alsip, San Diego, CA (US)

(72) Inventor: Murray Alsip, San Diego, CA (US)

(21) Appl. No.: 13/776,691

(22) Filed: Feb. 25, 2013

Related U.S. Application Data
(60) Provisional application No. 61/602,562, filed on Feb. 23, 2012.

(51) Int. Cl. A61B 1/267

(52) U.S. Cl. CPC A61B 1/267
USPC 600/197

(57) ABSTRACT

The present invention relates to a modified laryngoscope attachment device for use in “difficult” intubations and more specifically to a quick-attach power generator device that attaches to an existing laryngoscope to provide a means for a second user to help generate a power sufficient to open and/or maintain the opening of a “difficult” airway while not interfering with the first users ability to accurately guide the laryngoscope blade into its proper position. Embodiments of the present invention are directed to a power-generating handle adapted to be rapidly attached to the distal end of the handle of a laryngoscope to facilitate oral endotracheal intubation of a patient.

### CROSS REFERENCE TO RELATED APPLICATION(S)

This application claims the benefit of priority under 35 U.S.C. §119(e) of U.S. Ser. No. 61/602,562 filed Feb. 23, 2012 the entire content is incorporated herein by reference.

### BACKGROUND OF THE INVENTION

1. Field of the Invention

The invention generally relates to a laryngoscope including an attachable power generating handle and more specifically to a rapidly attached, non-articulating power generation handle secured to a laryngoscope.

2. Background Information

Field intubation by paramedics using direct laryngoscopy always carries the risk of the “difficult” intubation. Percentages of difficult to impossible intubation range as high as 10 percent and greater. (Chest, Jul. 30, 2010 Dr. Stewart, M.D., et al.) Numbers of impossible to intubate using direct laryngoscopy in the emergency department (ED) setting range as high as 1% or greater.

While a great deal of complex technology exists for intubation and airway management—many of these devices require technology that is not readily available in rural settings or in “field medicine.” These devices also require significant training and expertise. There are very few practical and effective and inexpensive devices that support the rescuer in a technique that they are already very experienced in, such as direct laryngoscopy. An advantage of the present invention is that it will require virtually no additional rescuer training.

One of the most important aspects of emergency care has been recognized as the ability for emergency personnel to manage a patient's airway. Reasons for difficult airway are many and well documented, including mallampati classification, thickness of the neck, overbite, obesity and numerous others. Despite all this data, the establishment of an endotracheal airway in a rapid fashion remains difficult and the inability to do so can sometimes result in death.

The most useful definition of a difficult airway is the inability to identify the appropriate anatomy (vocal cords) to be certain that the endotracheal tube is placed properly. If the vocal folds are partially identified or cannot be identified then this is termed a “blind airway.” Since the esophagus is right next to the cords and far more malleable it is far more likely to accidentally place the endotracheal tube in the esophagus. Thus proper visualization is the key—and this is aided by creating a slightly larger airway aperture (even by millimeters) and holding that aperture in position for the necessary amount of time to place an endotracheal (ET) tube between the vocal folds.

In performing an oral endotracheal intubation procedure the blade of a laryngoscope is inserted into the mouth of a patient. The tip of the blade is positioned at or near the epiglottis depending on whether a straight or curved laryngoscope blade is being used. A user manually gripped the handle of the laryngoscope with their left hand, and the single user manually gripped the handle of the laryngoscope with their left hand, and the single user lifted the laryngoscope along the central axis of the laryngoscope handle to lift the epiglottis and allow direct viewing of the vocal cords. While the single user gripped the handle of the laryngoscope with their left hand, the user inserted an endotracheal tube into the oral opening of the patient and into the larynx of the patient with the single users dominant right hand. Excessive torque or twisting of the laryngoscope blade when gripping the handle by the single user often resulted in undesired cranking or rotating of the laryngoscope which can cause trauma to the airway, broken or damaged teeth of the patient, and failed intubation attempts.

Although many difficult airway devices exist, most focus on technology for visualization, including fiber-optic devices. Virtually none address the problem at hand during a “difficult” intubation i.e., the inability to generate enough power to open a difficult or obstructed airway open enough and long enough temporally to visualize the vocal cords and insert an endotracheal tube.

Additionally, only one example of using the support of a second operator to provide additional power was identified in US Published Patent Application #2009/0299145 Titled: “Gear-Shaped Lifting Cap for a Laryngoscope”. This gear-shaped cap for a two-person power generating laryngoscope, has multiple shortcomings in a real-time trauma environment. First the Gear-shaped laryngoscope relies on a second person to grip a permanently affixed portion of the laryngoscope which has finger grooves to help tug and pull and direct. As will be discussed in the summary and detail of invention the present invention has found the ability of a second user to articulate the laryngoscope can be a hindrance to rapidly opening the airway. When two independent users are trying to guide a laryngoscope blade into the airway opening there is rarely pure conformity of movement, which can result in a negative effect. Additionally, the cap is made to replace the end of regular laryngoscopes, and therefore needs to be attached to a scope and ready to go even though in about 90% or more of the cases the cap will actually be distracting and a hindrance to “normal” or “non-difficult” intubations. Thus the prior art design is unwieldy and it is time consuming (probably takes at least a minute) to replace an end cap during the process of a difficult intubation, where seconds can have a dramatic effect on survival and/or life altering complications. Because of the essence of time, the prior art design would either need to be present on all scopes at all times or not be used at all during most difficult intubations where there is insufficient time to wait the minute or so it takes to remove the previous cap and add new modified cap.

Unfortunately, a power generating attachment that can be rapidly attached (in seconds rather than minutes) to an existing laryngoscope once a determination of a “difficult” intubation has been made, and that will not interfere with the first users ability to guide the laryngoscope blade into its proper position has not been described in the art. Thus a need exists for a quick-attach power generator device that attaches to an existing laryngoscope to provide a means for a second user to help generate a force sufficient to open a “difficult” airway while not interfering with the first users ability to accurately guide the laryngoscope blade into its proper position.

### SUMMARY OF THE INVENTION

When medical personnel are treating a patient, with an airway which requires intubation, speed and a proper visualization of the patients specific anatomy around the vocal cords is essential for proper intubation and patient survival. Most prior art has focused on visualization methods to see the orientation anatomy required for proper placement, but enhanced visualization techniques are not sufficient for removing the physical obstacles that block the actual insertion of the tube in “difficult” intubation cases. Typically, a single user has to try and provide enough power to open the obstructed pathway for a sufficient amount of time to allow while their non-dominant arm to provide a line of force and orientation that does not allow for the stronger arm muscles to control the process. This intubation technique results in a rapid fatigue of the non-dominant arm and oftentimes an inability of the medical personnel to remove the obstruction (typically the tongue or glottis) so that intubation can proceed.

An additional consideration particularly in the “field” is that the patients are not sedated or paralyzed as they are in a hospital setting, thus the speed and concert of movements against an often panicked or struggling subject becomes more essential.

The present invention relates to a modified laryngoscope for use in “difficult” intubations and more specifically to a quick-attach power generator device that attaches to an existing laryngoscope to provide a means for a second user to help generate a power sufficient to open a “difficult” airway while not interfering with the first users ability to accurately guide the laryngoscope blade into its proper position.

Embodiments of the present invention are directed to a power generating handle adapted to be rapidly (within seconds) attached to the distal end of the handle of a laryngoscope to facilitate oral endotracheal intubation of a patient.

Embodiments of the present invention include a handle attached to a quick release/attach clamp or affixation device for attaching the affixation component of the device to the distal end of a laryngoscope in a rapid and secure manner. Additional embodiments feature various handle and grip features for a second user to apply power in a linear way with minimal articulation abilities so that the second user does not interfere with the placement efforts of the first user.

Further embodiments feature the power generating handle attached to the distal end of the laryngoscope handle with various lengths of rod, cable, chain, or other inelastic material to provide the second user the opportunity to apply additional power on the laryngoscope to assist with opening the obstruction for a long enough amount of time while maintaining a distance so as not to interfere with the first users visual field and work space while concentrating on placing the laryngoscope blade in its proper orientation and ultimately while maintaining an unobstructed path for the intubation tube to be inserted by the first user.

Yet further embodiments feature various ways and materials for rapidly affixing the power generating device to the distal end of the laryngoscope including but not limited to: quick attach claims and clamping mechanisms such as c-clamps, tension clamps, zip-tie type attachments, hook and loop such as Velcro strap type attachments, and attachment ends meant to be held into position with adhesive tape.

Additionally, the embodiments of the present invention integrate with standard laryngoscopes and do not require modifications to existing laryngoscopes to function as specified.

### BRIEF DESCRIPTION OF THE DRAWINGS

The principles of the present invention will be apparent with reference to the following drawings, in which like reference numerals denote like components:

FIG. 1 is perspective view showing a prior art laryngoscope being manually grasped with the left hand of a sole user.

FIG. 2 is a diagrammatic view showing a prior art laryngoscope with a lifting cap for a second user assistance wherein the handle of the laryngoscope is grasped with a right hand of a first user, with the right hand of the first user in abutment with the proximal surface of the abutment member.

FIG. 3 is a diagrammatic view showing the prior art laryngoscope of FIG. 2 with a lifting cap for a second user assistance wherein the handle of the laryngoscope is grasped with a right hand of a first user, and a left hand of a second user is located in the finger-grip notches of the abutment member while the right hand of the firs user is being released from the handle of the laryngoscope.

FIG. 4 is a view of an embodied laryngoscope force generating device comprising FIGS. 4A-4E showing views of various surfaces. FIG. 4A shows a side elevation view of just an embodied device not yet attached to a laryngoscope. FIG. 4B shows a bottom plan view of said embodied device not yet attached to a laryngoscope. FIG. 4C shows a top perspective view of an embodied device attached to a laryngoscope. FIG. 4D shows a side elevation view of the embodied device attached to a laryngoscope. FIG. 4E shows a front elevational view of the embodied device attached to a laryngoscope.

FIG. 5 is a view of another embodied laryngoscope force generating device comprising FIGS. 5A-5D showing views of various surfaces. FIG. 5A shows a side elevation view of just an embodied device not yet attached to a laryngoscope. FIG. 5B shows a bottom plan view of said embodied device not yet attached to a laryngoscope. FIG. 5C shows a side elevation view of the embodied device attached to a laryngoscope. FIG. 5D shows a back elevational view of the embodied device attached to a laryngoscope.

FIG. 6 is an illustration showing a laryngoscope with an embodied quick attach force generating device contemplated in the present invention while in use on a patient. Wherein a second rescuer provides assistance by grasping the handle of the attachment device (with either their dominant or non-dominant hand) and the laryngoscope is grasped with the non-dominant hand of a first user.

FIG. 7 is a view of another embodied laryngoscope force generating device comprising FIGS. 7A-7D showing views of various surfaces. FIG. 7A shows a front elevation view of just an embodied device not yet attached to a laryngoscope. FIG. 7B shows a side elevation view of said embodied device not yet attached to a laryngoscope. FIG. 7C shows a side elevation view of the embodied device attached to a laryngoscope. FIG. 7D shows a back elevational view of the embodied device attached to a laryngoscope.

FIG. 8 is a view of yet another embodied laryngoscope force generating device comprising FIGS. 8A-8E showing views of various surfaces. FIG. 8A shows a side elevation view of just an embodied device not yet attached to a laryngoscope. FIG. 8B shows a front elevation view of said embodied device not yet attached to a laryngoscope. FIG. 8C shows a top perspective view of an embodied device not yet attached to a laryngoscope. FIG. 8D shows a side elevation view of the embodied device attached to a laryngoscope. FIG. 8E shows a back elevational view of the embodied device attached to a laryngoscope.

FIG. 9 is a side perspective view of another embodied rapidly attachable power generating device that is about to be placed onto a laryngoscope.

FIG. 10 comprises FIGS. 10A-10E which represent side perspective views of various alternative embodied rapid clamping type power generating devices attached to laryngoscopes.

### DETAILED DESCRIPTION OF THE INVENTION

The present invention contemplates either a modified laryngoscope which includes an attached power generating handle or a rapidly attachable power generating handle device capable of being attached to an existing laryngoscope as known in the prior art.

Most commonly difficult intubations are missed because the airway cannot be properly opened to visualize the vocal cords so the endotracheal tube can be seen to pass through them. This is termed a “blind” intubation where the vocal cords are not visualized or are poorly visualized. The natural anatomy of the neck is such that blind intubations usually result in esophageal intubations ad are thus “missed.”

Embodiments of the present invention are designed to decrease the number of “missed” intubations by giving medical personnel a better device for not only visualizing the vocal cords in difficult airway situations but also by providing a more open path for the endotracheal tube to travel. The modified laryngoscopes or quick attach laryngoscope power generating devices contemplated by the present invention are designed to improve the visualization and pathway of the endotracheal tube by allowing a concert of movement between a first user who then may direct a second to apply power as needed to open or maintain an endotracheal tube passageway until a complete and successful airway is maintained.

Embodiments of the present invention are for a rapidly attachable laryngoscope handle which allows rescuers to generate more power than typically capable when using a standard laryngoscope without the power generating handle. The embodiments refer to power and not force because the ability to generate more power is essential to the device. The desire is to have the rapidly attachable handles assist with generating more power by generating enough force to open the aperture of the mouth a few more millimeters and/or hold it for a greater duration of time to allow a rescuer to clearly visualize the cords as the rescuer places the endotracheal tube between the cords.

Definitions

Definition of terms as defined by the American Society of Anesthesiologists (ASA).

Difficult Airway: the clinical situation in which a conventionally trained anesthesiologist experiences difficulty with mask ventilation, difficulty with tracheal intubation, or both.

Difficult laryngoscopy: not being able to see any part of the vocal cords with conventional laryngoscopy.

Difficult intubation: proper insertion with conventional laryngoscopy requires either (1) more than three attempts or (2) more than ten minutes. A difficult intubation determination can also be determined after 1 attempt and within 30 seconds.

Failed intubation: the inability to place an endotracheal tube.

Additional definitions:

Rapidly attachable handle: is defined as a handle capable of being attached to the handle of a standard laryngoscope in less than a minute, preferably less than 30 seconds and most preferably in less than 15 seconds by a typical user.

Generating Power: Power is defined as Force×Duration. Thus P=F×D. For the purpose of the invention the power equal to the amount of force necessary to open an obstructed airway for a duration sufficient to allow the user to insert an endotracheal tube to establish an airway for a patient. Thus allowing the primary rescuer to provide more force with a greater airway aperture and hold that position for a greater time so that appropriate anatomic structures can be identified (the vocal cords) and the endotracheal tube can be safely placed.

Illustrative embodiments of the invention are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming, but would nevertheless be a routing undertaking for those of ordinary skill in the art having the benefit of this disclosure.

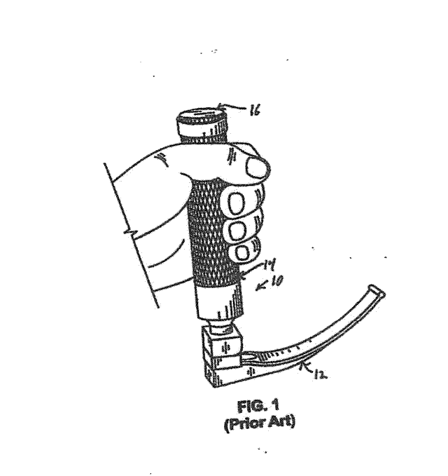

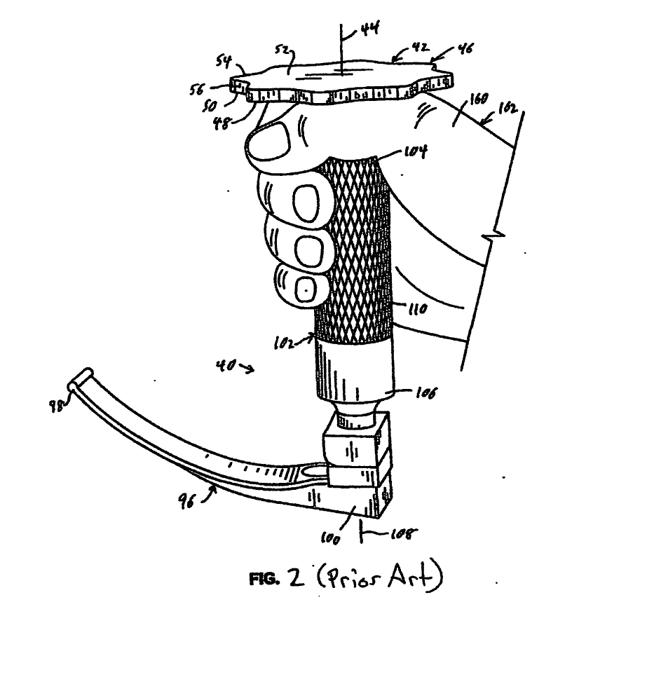

FIG. 1 shows a prior art laryngoscope 10 being manually grasped by the (non-dominant) left hand of a single user. The laryngoscope 10 includes a blade 12 that is attached to a generally cylindrical handle 14. Handle 14 is often tubular such that it may contain one or more batteries. The laryngoscope 10 includes a threaded end cap 16 that is removably attached to an internally threaded distal end of handle 14. End cap 16 has a diameter that is approximately equal to the diameter of handle 14.

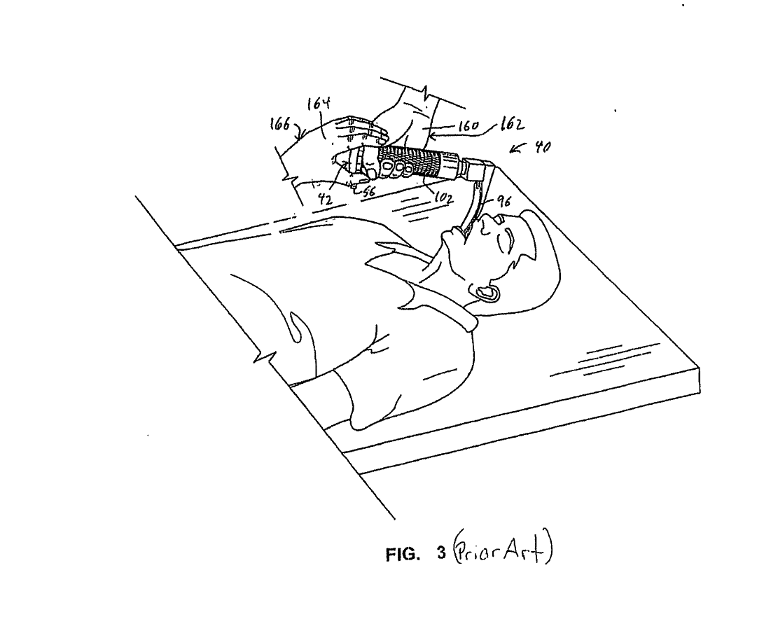

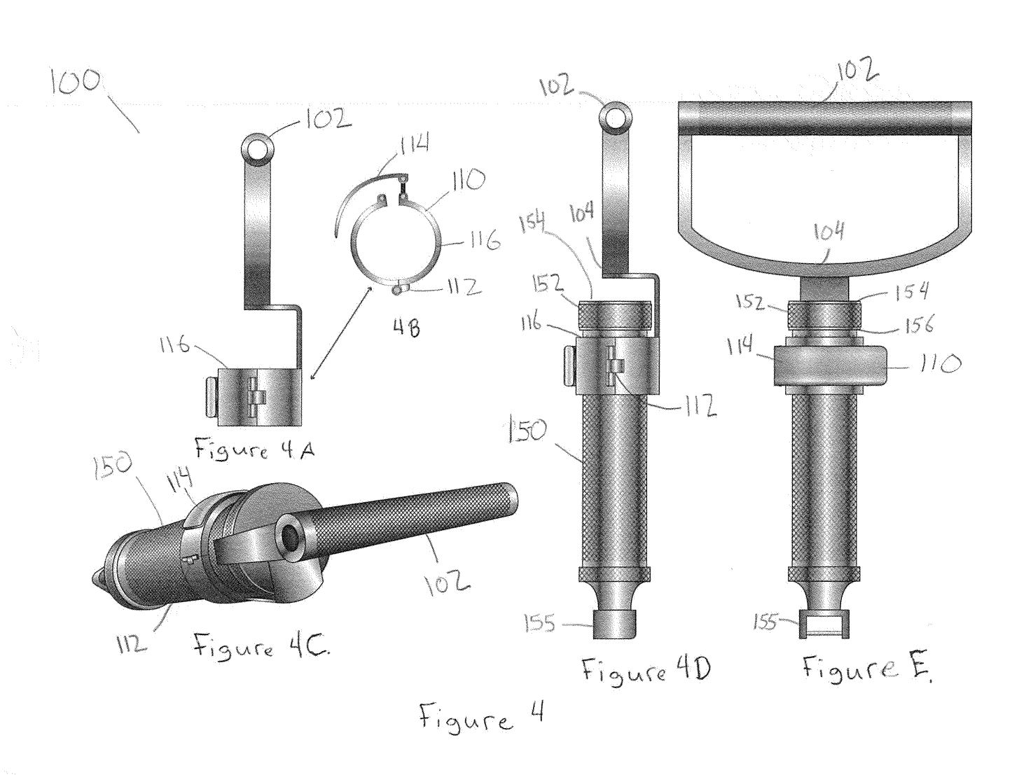

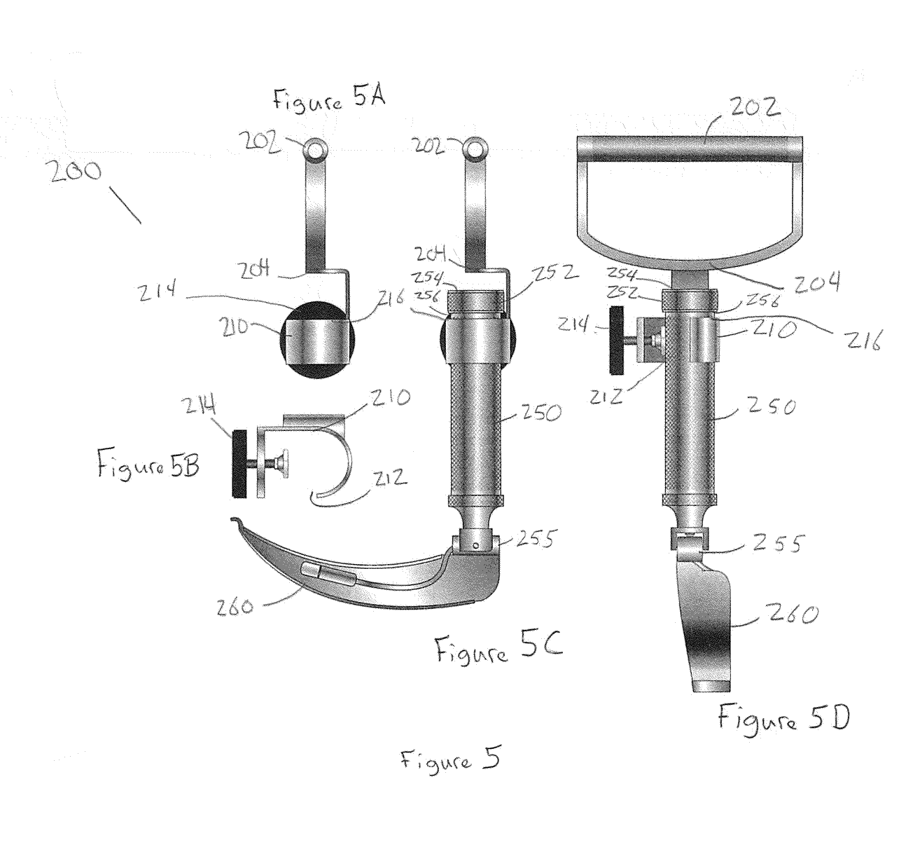

FIGS. 2 and 3 show a prior art laryngoscope with an attached lifting cap wherein the handle is grasped by the dominant hand of a first rescuer and a second rescuer grasps the lifting cap and maintains the laryngoscope in place after the laryngoscope has been placed in its proper orientation. FIG. 3 is a diagrammatic view showing the prior art laryngoscope of FIG. 2 with a lifting cap for a second user assistance wherein the handle of the laryngoscope is grasped with a right hand of a first user, and a left hand of a second user is located in the finger-grip notches of the abutment member while the right hand of the firs user is being released from the handle of the laryngoscope.

FIG. 4 is a series of drawings of an embodied laryngoscope power generating system 100 comprising FIGS. 4A-4E showing various views of an embodied device. FIG. 4A shows a side elevation view of just an embodied device 100 not yet attached to a laryngoscope 150. FIG. 4A shows the power handle 102 to be attached to a laryngoscope handle 150 and controlled by the second rescuer under the direction of the first rescuer. The embodied laryngoscope attachment device 100 in FIG. 4 comprises a power handle 102 that has a power handle end portion 104 which is attached to a power handle attachment mechanism 110. The power handle attachment mechanism 110 is designed to be secured around a laryngoscope handle 150 and includes an opening mechanism 112 which allows the attachment mechanism 110 to be opened up and placed around the handle of a laryngoscope, then a tightening/closing mechanism 114 is engaged to secure the attachment mechanism 110 and the device 100 to the laryngoscope handle 150. As illustrated in FIG. 4B the attachment mechanism 110 further includes a top ridge 116 which may abut against the bottom edge 156 of the laryngoscope cap 152. The closing/tightening mechanism 114 of the embodied device 100 shown is similar to a bicycle seat tightening mechanism known in the art. FIGS. 4C-4E show the embodied device 100 attached to a laryngoscope handle 150. When attached, the power attachment mechanism 110 is secured around the laryngoscope handle 150 distal from the laryngoscope blade 155 and immediately abutting or proximal to the laryngoscope cap 152. The bottom surface 156 of the laryngoscope cap 152 may abut against the top surface 116 of the power attachment mechanism 110 to further secure the device 100 from sliding off of the laryngoscope. The top surface of the cap 154 in this embodiment is unaltered and the embodied power attachment device 100 is designed to mate with a non-modified laryngoscope or cap. The embodiments of the present invention are designed such that the interface of the device with a second rescuer/assistant is under the direction of the first rescuer who directs the concert of movement and power generated by the two rescuers to properly insert an endotracheal tube.

FIG. 5 is a series of drawings of another embodied laryngoscope power generating system 200 comprising FIGS. 5A-5D showing various views of the embodied device 200. FIG. 4A shows a side elevation view of just an embodied power device 200 not yet attached to a laryngoscope 250. FIG. 5A shows the power handle portion 202 to be attached to a laryngoscope handle 250 and controlled by the second rescuer under the direction of the first rescuer. The embodied laryngoscope attachment device 200 in FIG. 5 comprises a power handle 202 that has a power handle end 204 which is attached to a power handle attachment mechanism 210. The power handle attachment mechanism 210 is designed to be secured around a laryngoscope handle 250 and includes an opening mechanism 212 which allows the power attachment mechanism 210 to be opened up and placed around the handle of a laryngoscope, then a tightening/closing mechanism 214 is engaged to secure the attachment mechanism 210 and the device 200 to a laryngoscope handle. As illustrated in FIG. 5C the attachment mechanism 210 further includes a top ridge 216 which may abut against the bottom edge 256 of the laryngoscope cap 252. The closing/tightening mechanism 214 of the embodied device 200 shown is a tightening screw mechanism known in the art. FIGS. 5C and 5D show the embodied device 200 attached to a laryngoscope handle 250. When attached the attachment mechanism 210 is secured around the laryngoscope handle 250 distal from the laryngoscope blade 255 and immediately abutting or proximal to the laryngoscope cap 252. The bottom surface 256 of the laryngoscope cap 252 may abut against the top surface 216 of the attachment mechanism 210 to further secure the device 200 from sliding off of the laryngoscope. The top surface of the cap 254 in this embodied is unaltered and the embodied attachment device 200 is designed to mate with a non-modified laryngoscope or cap. The embodiments of the present invention are designed such that the interface of the device with a second rescuer/assistant is under the direction of the first rescuer who directs the concert of movement and power generated by the two rescuers to properly insert an endotracheal tube.

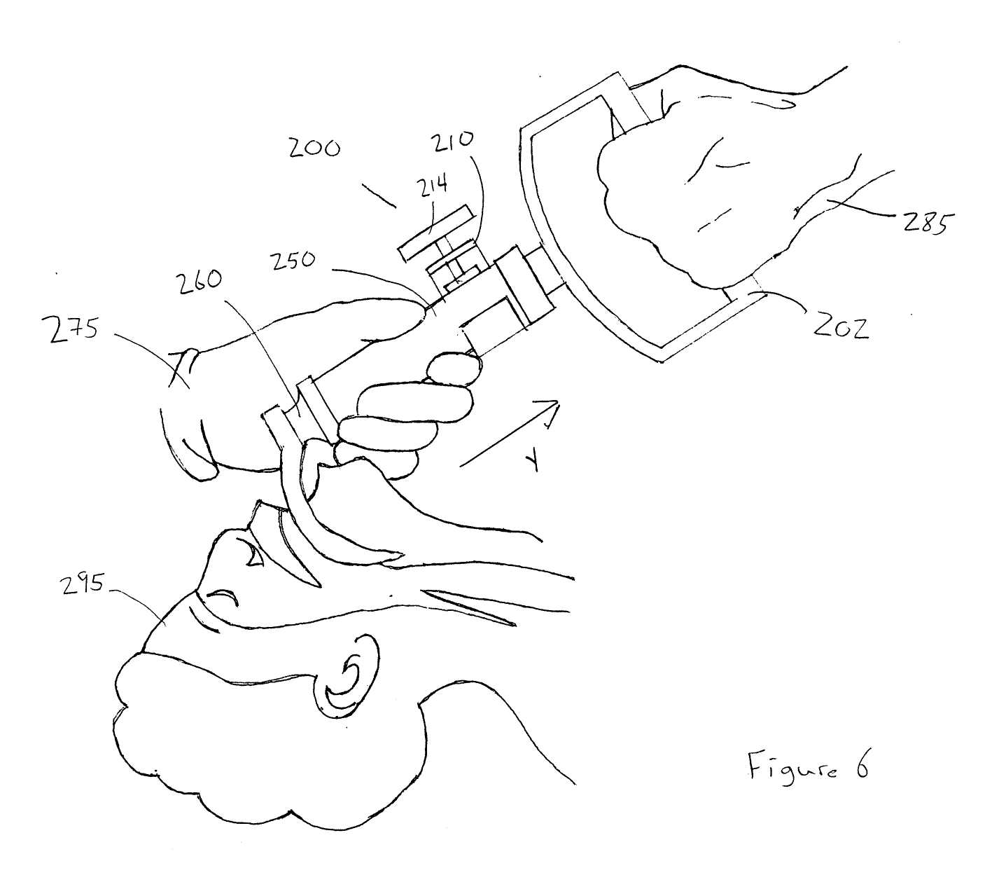

FIG. 6 demonstrates the use of the embodied power generating handle mechanism 200 shown in FIGS. 5A-5D. In the illustration of FIG. 6 the power generating handle 200 is designed to be controlled by the second rescuer 285 without interfering with the first rescuers 275 ability to control the laryngoscope 250 or to interfere with the first rescuers 275 ability to intubate the patient 295 in any way. The second rescuers 285 job is to use the embodied rapidly attachable power generating handle 200 to generate power by generating force in a opening direction y to further open the airway and/or to hold the airway open for a duration of time necessary for proper visualization and correct intubation to occur. Thus, if a small amount of force is needed then the second user is directed to provide a small amount of force by pulling the power handle 202 in the proper direction. Likewise if the first rescuer 275 needs the opening maintained for a duration of time necessary for the first rescuer 275 to properly visualize the cords, then the second rescuer 285 is instructed to hold the laryngoscope blade 260 in a fixed position and generate the power associated with the mechanism 200 until the proper insertion of the endotracheal tube is completed. The embodiment of mechanism 200 is designed such that the interface of the device 200 with a second rescuer/assistant is under the direction of the first rescuer who directs the concert of movement and power generated by the two rescuers to properly insert an endotracheal tube.

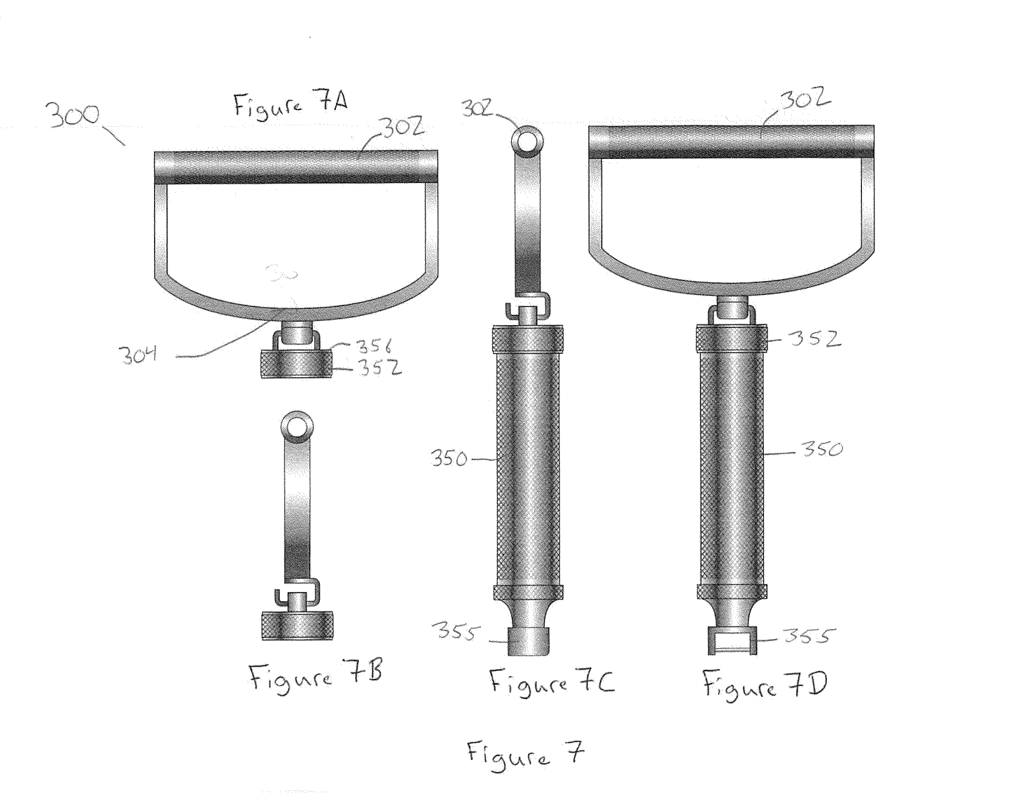

FIG. 7 is a series of drawings of yet another embodied laryngoscope power generating system 300 comprising FIGS. 7A-7D showing various views of the embodied device 300. FIG. 7A shows a front elevation view of just the embodied power device 300 not yet attached to a laryngoscope 350. FIG. 7A shows the power handle portion 302 to be attached to a laryngoscope handle 350 and controlled by the second rescuer under the direction of the first rescuer. The embodied laryngoscope attachment device 300 in FIG. 7 comprises a power handle 302 that has a power handle end 304 which is attached to a power handle attachment mechanism 310. The power handle attachment mechanism 310 is designed to mate with an extension 358 on the top surface 356 of a laryngoscope cap 352 and then secured to a laryngoscope handle 350. As illustrated in FIG. 7C the power attachment is based on the hooking of the power handle attachment mechanism 310 around the extension 358 of the laryngoscope cap. FIGS. 7C and 7D show the embodied device 300 attached to a laryngoscope handle 350. The top surface 356 of the cap 352 in this embodiment is altered and the embodied attachment device 300 is thus designed to mate with a modified laryngoscope or cap. However the modification is minor and unobtrusive and allows a rescuer to rapidly hook the embodied power generator 300 unto a laryngoscope previously fitted with the special cap 352.

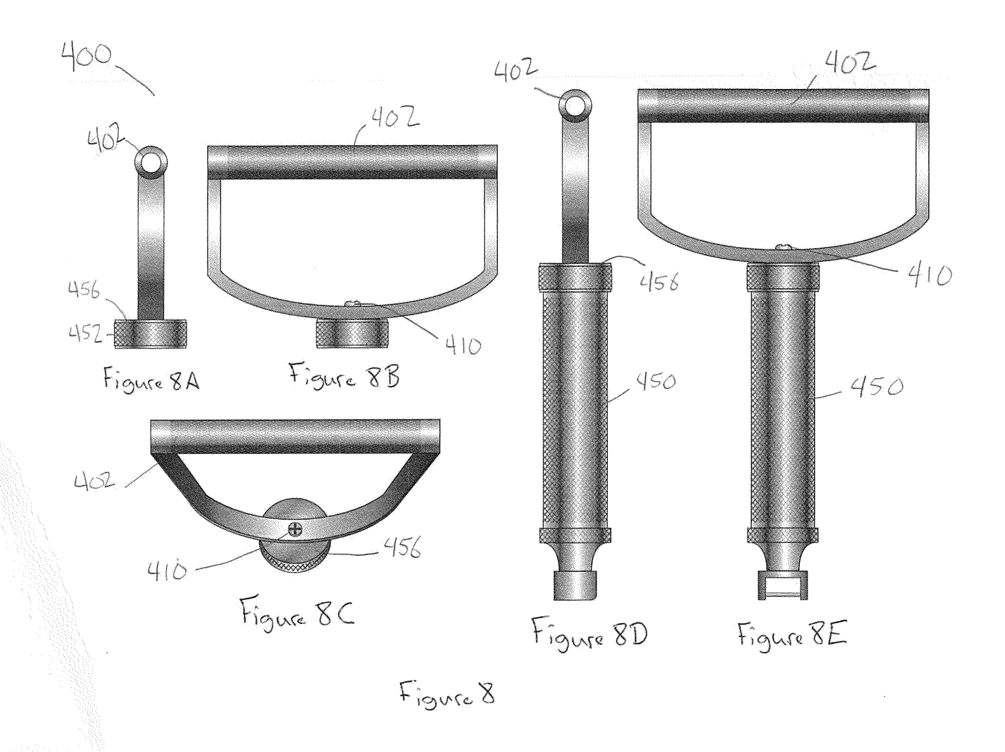

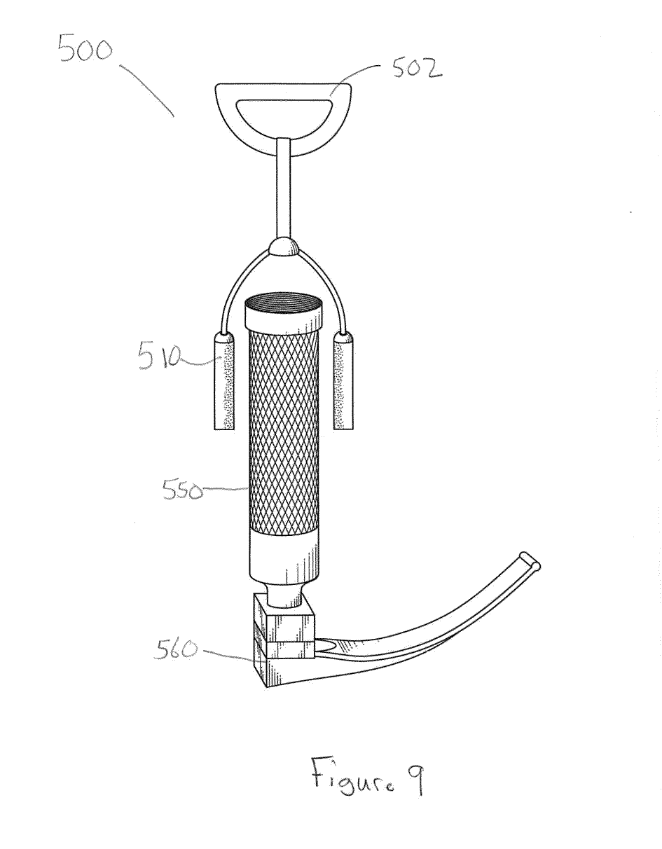

FIG. 8 is a series of drawings of still yet another embodied laryngoscope power generating system 400 comprising FIGS. 8A-8E showing various views of the embodied device 400. FIG. 8A shows a side elevation view of just the embodied power device 400 not yet attached to a laryngoscope 450. FIGS. 8A, 8B and 8C shows the power handle portion 402 to be attached to a laryngoscope handle 450 and controlled by the second rescuer under the direction of the first rescuer. The embodied laryngoscope attachment device 400 in FIG. 8 comprises a power handle 402 that has a power handle end 404 which is attached to a power handle attachment mechanism 410. The power handle attachment mechanism 410 is a screw designed to mate with intrusion portion (threads) 458 that open up on the top surface 456 of a laryngoscope cap 452 and extend within the cap 452, and wherein the cap 452 is secured to a laryngoscope handle 450. As illustrated in FIG. 8D the power attachment is based on the screwing of the power handle attachment mechanism 410 into the intrusion portion 458 of the laryngoscope cap 452. FIGS. 8D and 8E show the embodied device 400 attached to a laryngoscope handle 450. The top surface 456 of the cap 452 in this embodiment is altered and the embodied attachment device 400 is thus designed to mate with a modified laryngoscope or cap. However the modification is minor and unobtrusive and allows a rescuer to rapidly screw the embodied power generator 300 into a laryngoscope cap previously fitted with the special cap 352.

In FIG. 9 another power generating mechanism 500 is shown with different attachment mechanism 510 that comprises with magnetic attachment straps, or regular straps 510 that are secured to the laryngoscope handle 550 rapidly with tape, or held onto by the first user and secured merely by the grip of the first rescuer against the side of the laryngoscope handle.

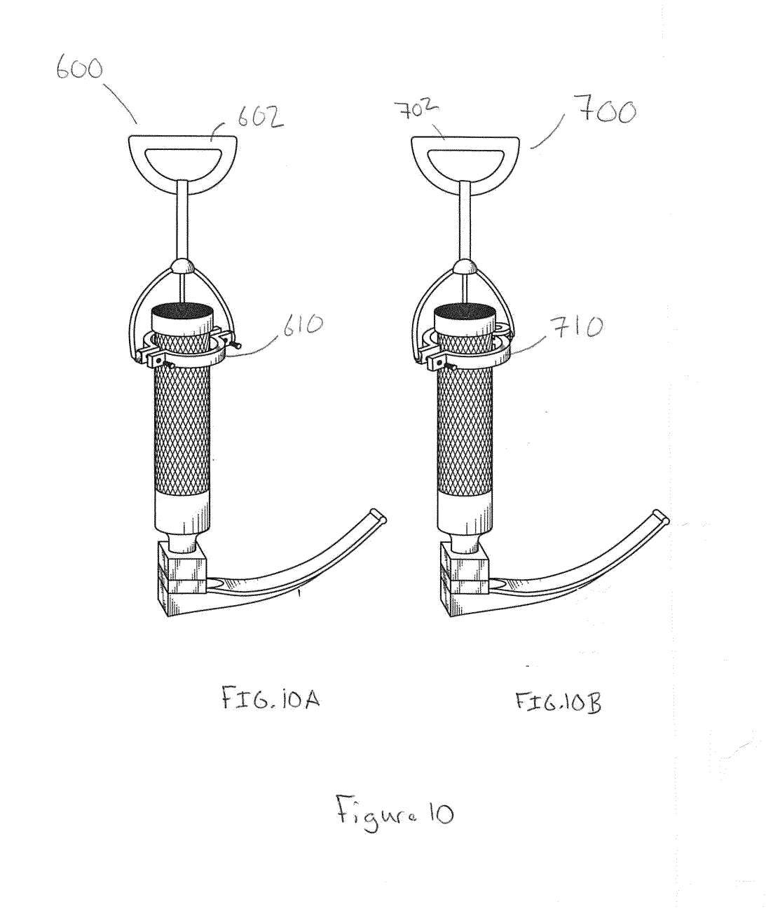

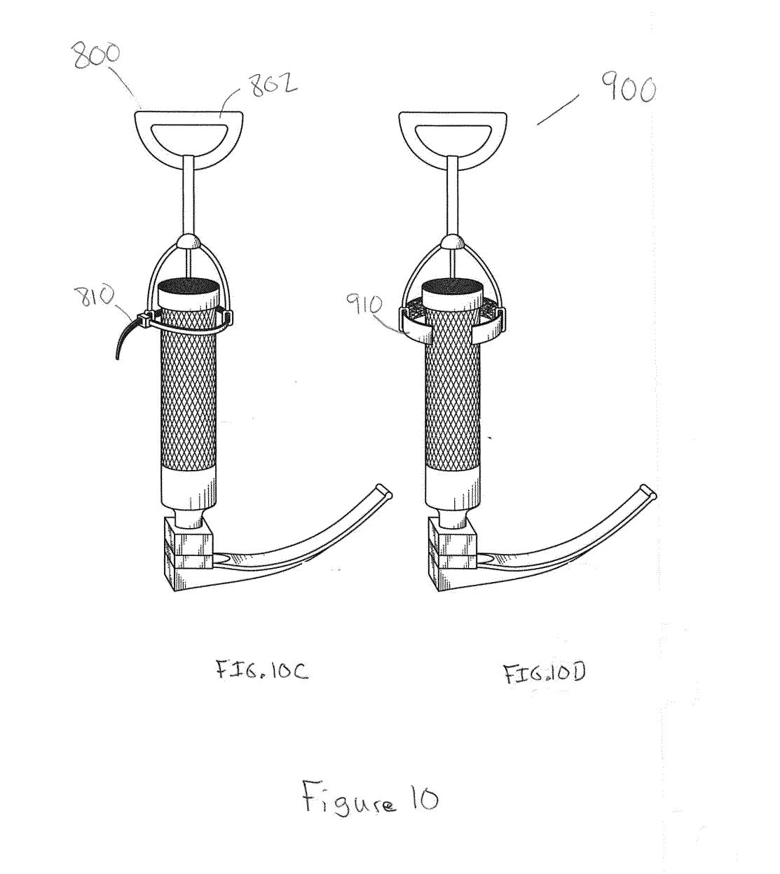

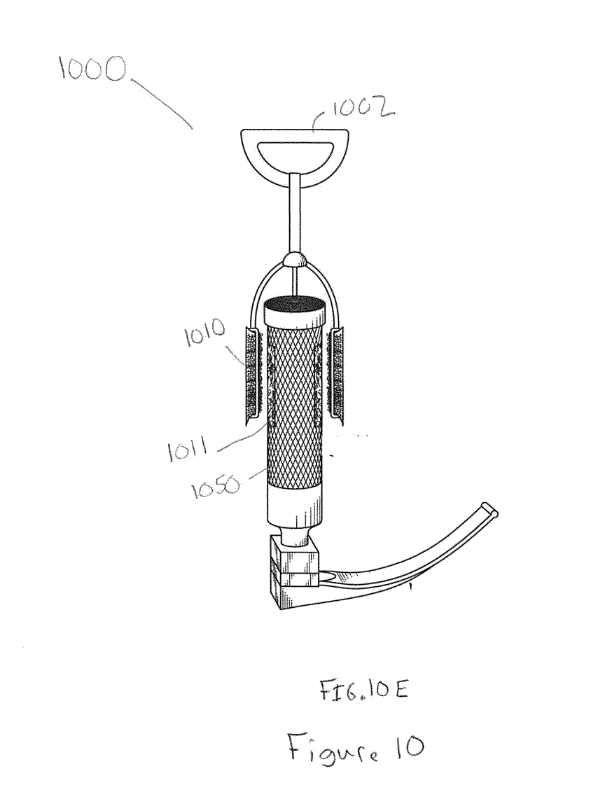

FIG. 10 comprises FIGS. 10A-10E which all represent alternative embodiments of the present invention. FIG. 10A shows an embodied device 600 wherein the power attachment mechanism 610 is a c-clamp which may be secured to the handle of an existing laryngoscope. FIG. 10B shows an embodied device 700 wherein the power attachment mechanism 710 is in the form of a hinge-clamp type device. Additional clamping devices known in the art are contemplated in the present invention. FIG. 10C shows an embodied device 800 wherein the power attachment mechanism 810 is in the form of a zip-tie type feature wherein the handle can be secured onto the laryngoscope with one or more zip-tie type fasteners commonly known in the art. FIG. 10D shows an embodied device 900 wherein the power attachment mechanism 910 is in the form of an attachment band or hook and loop system. FIG. 10E shows an embodied device 1000 wherein the power attachment mechanism 1010 is in the form of a hook and loop type fastening system such as Velcro. The attachment mechanisms shown above may have additional lip abutment features further decreasing the ability of the device to slide off the laryngoscope.

Although the invention has been described with reference to the above examples, it will be understood that modifications and variations are encompassed within the spirit and scope of the invention. Accordingly, the invention is limited only by the following claims.

What is claimed is:

1. A power generating handle adapted to be rapidly attached to a distal end of the handle of a laryngoscope to facilitate oral endotracheal intubation of a patient;
wherein the power generating handle is attached to a quick release attach clamp or affixation component for attaching the affixation component of the device to the distal end of a laryngoscope in a rapid and secure manner.

2. A medical device for generating power when used in conjunction with a laryngoscope comprising:
a power generating handle intended to be used by a second rescuer, and
an attachment means for rapidly securing a handle to an existing laryngoscope.

3. The device of claim 2 wherein the power generating handle can be secured to a laryngoscope handle in less than a minute.

4. The device of claim 3 wherein the power generating handle can be secured to a laryngoscope handle in less than 15 seconds on average by a rescuer in a hospital or field setting.

5. The medical device for assisting with difficult intubations comprising:
a power generating attachment handle configured to be attached to a standard laryngoscope;
wherein the power generating attachment handle comprises an attachment means which attaches the mechanism to a handle of said larnyngoscope;
wherein the attachment handle mechanism is designed to attach to a handle rapidly in emergency conditions;
wherein the power generating handle enable a second rescuer to assist with opening an airway that is obstructed and/or maintain an airway open long enough for a first rescuer to intubate a patient.
